(19) United States
(12) Patent Application Publication
(10) Pub. No.: US 2025/0352590 A1
(43) Pub. Date: Nov. 20, 2025

(54) Functional postbiotic composition for homeostatic vaginal care

(71) Applicant: Seed Health Inc., Venice, CA (US)

(72) Inventor: Tye Jensen, Telluride, CO (US)

(21) Appl. No.: 19/213,405

(22) Filed: May 20, 2025

Related U.S. Application Data
(60) Provisional application No. 63/649,797, filed on May 20, 2024.

(51) Int. Cl. A61K 35/747; A61K 31/191; A61K 31/7016; A61P 15/02; A61P 31/04; A61P 31/10

(52) U.S. Cl. CPC A61K 35/747; A61K 31/191; A61K 31/7016; A61P 15/02; A61P 31/04; A61P 31/10

(57) ABSTRACT

Postbiotic compositions for maintenance and/or restoration of a healthy vaginal environment and/or microbiome are disclosed. The postbiotic compositions include a paraprobiotic component, comprising inactivated cells of Lactobacillus crispatus, and a metabolite component, comprising D-lactate.

### CROSS-REFERENCE TO RELATED APPLICATION

This application claims the benefit of priority under 35 U.S.C. § 119 (e) to U.S. Provisional Patent Application 63/649,797, filed 20 May 2024, the entirety of which is incorporated herein by reference.

### FIELD

The present disclosure relates to postbiotic compositions, and particularly to postbiotic compositions for maintenance of homeostasis of the vaginal environment that include inactivated and/or nonviable Lactobacillus crispatus cells and D-lactate.

### BACKGROUND

Postbiotics generally, and paraprobiotics in particular, are gaining traction in recent years due to concerns about the possibility of low tolerance of probiotics, especially in pediatric populations and in severely ill or immunocompromised patients. In some cases, paraprobiotics may have beneficial properties similar to live probiotics, with fewer of the constraints associated with live bacteria (e.g., poor shelf stability, difficulty and/or cost of manufacture, etc.). The mechanisms of action of paraprobiotics are not well understood, but may include immune system regulation and interference with pathogen attachment to host cells; some research hypothesizes that paraprobiotics may release key bacterial components, such as lipoteichoic acids, peptidoglycans, or exopolysaccharides, which may exhibit immunomodulating effects and/or act as antagonists against pathogens.

To date, the great majority of research into postbiotics, and especially paraprobiotics, has focused on modulation of the gut microbiome by administration of oral postbiotic formulations. While some limited attention has been given to the potential use of oral and/or topical postbiotic formulations in modulating the skin microbiome, many other potential applications for postbiotics, such as modulation of the vaginal microbiome, remain largely unexplored.

The vaginal microbiome differs in important ways from other microbiomes; for example, while an optimal gut microbiome is a highly diverse, high-biomass microbial community, an optimal vaginal microbiome is characterized by low bacterial diversity often dominated by one species of Lactobacillus. Metataxonomic studies utilizing 16S rRNA gene sequencing analysis have revealed that there are five major Community State Types (CSTs) of the vaginal microbiome, of which four are dominated by one species of Lactobacillus: CST I (dominated by L. crispatus), CST II (dominated by L. gasseri), CST III (dominated by L. iners), and CST V (dominated by L. jensenii). CST IV, however, which includes the vaginal microbiomes of about 25% of women, is characterized by a relative dearth of Lactobacillus spp. Low abundance of Lactobacillus in the vaginal microbiome is associated with increased risk for severe adverse gynecologic and obstetric outcomes. Adverse gynecologic outcomes associated with low Lactobacillus abundance include, but are not limited to, acquisition of sexually transmitted infections (STIs) (including human immunodeficiency virus (HIV), chlamydia, gonorrhea, herpes simplex virus (HSV), and human papillomavirus (HPV)), bacterial vaginosis (the most frequently cited cause of vaginal discharge and malodor), yeast infection, urinary tract infection (UTI), and pelvic inflammatory disease (PID). Adverse obstetric outcomes associated with low Lactobacillus abundance include, but are not limited to, preterm delivery and low birth weight, infertility, stillbirth, premature rupture of membranes (PROM), postpartum and postabortal endometritis, amniotic fluid infection, and chorioamnionitis. Lactobacillus spp. are thus key to reproductive and gynecological health, and not all CSTs are equally protective; CST IV is associated with high risk to these and other adverse health outcomes, and CST III is suboptimal compared to CSTs I, II, and V. Moreover, it is known that the distribution of vaginal microbiome CSTs varies with race; for example, 40.5% of black women and 38.1% of Hispanic women harbor a CST IV vaginal microbiome, compared to 19.8% of Asian women and 10.3% of white women. While several proposed solutions to the restoration and maintenance of vaginal microbiota associated with positive health outcomes exist, these proposed solutions are typically probiotic, i.e., they rely on administration of live bacteria (typically live Lactobacillus spp. bacteria), which for the reasons explained above may in some cases be less desirable than a paraprobiotic or other postbiotic approach. Even in applications where a probiotic composition is useful and desirable, the inclusion of a significant quantity of inactivated cells, cell lysates, etc. to form a combination probiotic/postbiotic product may have additional advantages and benefits relative to a probiotic composition alone.

There is thus a need in the art for paraprobiotics and other postbiotics that maintain and/or restore homeostasis in, or otherwise provide a physiological benefit to, the vaginal environment, microbiome, and/or tissues.

### SUMMARY

In an aspect of the present disclosure, a postbiotic composition comprises a paraprobiotic component, comprising inactivated cells of Lactobacillus crispatus; and a metabolite component, comprising D-lactate in an amount of at least about 5 milligrams per gram of the postbiotic composition.

In some embodiments, the postbiotic composition further comprises a probiotic component.

In some embodiments, the probiotic component comprises live cells of Lactobacillus crispatus.

In some embodiments, at least a portion of the inactivated cells of Lactobacillus crispatus in the paraprobiotic component are of the same strain or consortium of strains as at least a portion of the live cells of Lactobacillus crispatus in the probiotic component.

In some embodiments, the inactivated cells of Lactobacillus crispatus in the paraprobiotic component, the live cells of Lactobacillus crispatus in the probiotic component, or both comprise cells of at least one strain of Lactobacillus crispatus selected from the group consisting of LUCA111 (ATCC Accession Deposit No. PTA-127219), LUCA011 (ATCC Accession Deposit No. PTA-127214), LUCA015 (ATCC Accession Deposit No. PTA-127215), LUCA009 (ATCC Accession Deposit No. PTA-127213), LUCA102 (ATCC Accession Deposit No. PTA-127217),

LUCA006 (ATCC Accession Deposit No. PTA-127211), LUCA059 (ATCC Accession Deposit No. PTA-127216), LUCA103 (ATCC Accession Deposit No. PTA-127218), and LUCA008 (ATCC Accession Deposit No. PTA-127212).

In some embodiments, at least a portion of the D-lactate in the metabolite component is produced as a product or byproduct of a metabolic process of live Lactobacillus crispatus cells.

In some embodiments, at least a portion of the live Lactobacillus crispatus cells that produce the at least a portion of the D-lactate in the metabolite component are subsequently inactivated and form at least a portion of the inactivated Lactobacillus crispatus cells of the paraprobiotic component.

In some embodiments, substantially all of the D-lactate in the metabolite component is produced as a product or byproduct of a metabolic process of live Lactobacillus crispatus cells.

In some embodiments, a ratio of the D-lactate produced as a product or byproduct of a metabolic process of live Lactobacillus crispatus cells to added lactate is from about 2:5 to about 2:1.

In some embodiments, the added lactate is selected from the group consisting of lactate salts, lactic acid, alkyl lactates, and combinations thereof.

In some embodiments, the postbiotic composition further comprises maltose.

In another aspect of the present disclosure, a therapeutic formulation comprises a postbiotic composition as disclosed herein; and a pharmaceutically acceptable vehicle.

In some embodiments, the therapeutic formulation is configured for intravaginal administration.

In some embodiments, the therapeutic formulation is configured for topical administration.

In some embodiments, the therapeutic formulation is configured to be effective, when administered to a human subject, to treat, prevent, or reduce the likelihood of a disease, disorder, condition, or symptom in the subject, wherein the disease, disorder, condition, or symptom is selected from the group consisting of bacterial vaginosis, pelvic inflammatory disease, sexually transmitted infection, postpartum endometritis, postpartum sepsis, preterm birth, preterm premature rupture of membranes, miscarriage, chorioamnionitis, intra-amniotic infection, infertility, ineffectiveness of infertility treatment, cervical intraepithelial neoplasia, cervical cancer, another cancer of the female genitourinary system, and combinations thereof.

Another aspect of the present disclosure includes a unit dosage form of a therapeutic formulation as disclosed herein.

In some embodiments, the unit dosage form comprises from about 1 billion to about 100 billion inactivated Lactobacillus crispatus cells.

In some embodiments, the unit dosage form comprises from about 6 mg to about 20 mg of D-lactate.

In another aspect of the present disclosure, a method for treating, preventing, or reducing the likelihood of a disease, disorder, condition, or symptom in a human subject comprises administering to the subject a therapeutically effective amount of a postbiotic composition as disclosed herein.

In some embodiments, the disease, disorder, condition, or symptom is selected from the group consisting of bacterial vaginosis, pelvic inflammatory disease, sexually transmitted infection, postpartum endometritis, postpartum sepsis, preterm birth, preterm premature rupture of membranes, miscarriage, chorioamnionitis, intra-amniotic infection, infertility, ineffectiveness of infertility treatment, cervical intraepithelial neoplasia, cervical cancer, another cancer of the female genitourinary system, and combinations thereof.

In some embodiments, the postbiotic composition is administered intravaginally or topically to external surfaces of the vulva of the subject.

In some embodiments, a unit dosage form of the postbiotic composition comprises from about 1 billion to about 100 billion inactivated Lactobacillus crispatus cells.

In some embodiments, a unit dosage form of the postbiotic composition comprises from about 6 mg to about 20 mg of D-lactate.

In some embodiments, a postbiotic composition as disclosed herein is suitable for use in a method of treating the human or animal body by therapy.

In some embodiments, a postbiotic composition as disclosed herein is suitable for use in a method of treating, preventing, or reducing the likelihood of a disease, disorder, condition, or symptom selected from the group consisting of bacterial vaginosis, pelvic inflammatory disease, sexually transmitted infection, postpartum endometritis, postpartum sepsis, preterm birth, preterm premature rupture of membranes, miscarriage, chorioamnionitis, intra-amniotic infection, infertility, ineffectiveness of infertility treatment, cervical intraepithelial neoplasia, cervical cancer, another cancer of the female genitourinary system, and combinations thereof.

Another aspect of the present disclosure includes the use of a postbiotic composition as disclosed herein for the preparation of a medicament for treating, preventing, or reducing the likelihood of a disease, disorder, condition, or symptom.

In some embodiments, the disease, disorder, condition, or symptom is selected from the group consisting of bacterial vaginosis, pelvic inflammatory disease, sexually transmitted infection, postpartum endometritis, postpartum sepsis, preterm birth, preterm premature rupture of membranes, miscarriage, chorioamnionitis, intra-amniotic infection, infertility, ineffectiveness of infertility treatment, cervical intraepithelial neoplasia, cervical cancer, another cancer of the female genitourinary system, and combinations thereof.

In another aspect of the present disclosure, a method for manufacturing a postbiotic composition comprises culturing Lactobacillus crispatus cells in a fermentation medium, whereby the Lactobacillus crispatus cells produce D-lactate as a product or byproduct of a metabolic process; separating at least a portion of the Lactobacillus crispatus cells and at least a portion of the D-lactate from the fermentation medium; inactivating the separated Lactobacillus crispatus cells; and combining the inactivated Lactobacillus crispatus cells and the separated D-lactate to form the postbiotic composition.

In some embodiments, the inactivating step comprises at least one of heat inactivation, ultraviolet inactivation, chemical treatment, gamma irradiation, sonication, and tabletization.

In some embodiments, in the combining step, the inactivated Lactobacillus crispatus cells and the separated D-lactate are further combined with an added source of lactate.

In some embodiments, an amount of lactate provided by the added source of lactate is about 50% to about 250% of the amount of the separated D-lactate.

In some embodiments, the added source of lactate is selected from the group consisting of lactate salts, lactic acid, alkyl lactates, and combinations thereof.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIGS. 1A, 1B, and 1C are graphs of D-lactate concentration in a culture medium during fermentation of Lactobacillus crispatus strains LUCA009, LUCA011, and LUCA103, respectively.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

As used herein, unless otherwise specified, the term “animal” refers to any organism of the kingdom Animalia, including but not limited to a human.

As used herein, unless otherwise specified, the term “disease” refers to a disease, disorder, or condition, or a symptom thereof.

As used herein, unless otherwise specified, the term “paraprobiotic” refers to any formulation of inactivated and/or nonviable bacterial cells, or any fractions, fragments, or lysates thereof, that, when administered to a subject in a therapeutically effective amount, provides a physiological benefit (e.g., triggers a positive biological response, maintains or restores homeostasis, etc.) to the subject.

As used herein, unless otherwise specified, the terms “patient” and “subject” are interchangeable and each refer to a mammal, including, by way of non-limiting example, a human.

As used herein, unless otherwise specified, the term “pharmaceutically acceptable” means approved or approvable by a governmental drug or medicine regulator, such as the United States Food and Drug Administration or the European Medicines Agency, or listed in the United States Pharmacopoeia or other generally recognized pharmacopoeia for use in humans specifically or mammals or animals more broadly.

As used herein, unless otherwise specified, the term “pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a substance provided by the present disclosure may be administered to a patient, which does not destroy the pharmacological activity thereof, and which is non-toxic when administered in doses sufficient to provide a therapeutically effective amount of the substance.

As used herein, unless otherwise specified, the term “postbiotic” refers to any substance that (1) provides a physiological benefit (e.g., triggers a positive biological response, maintains or restores homeostasis, etc.) to a subject when administered to the subject in an effective dosage amount, and (2) is at least one of (a) a paraprobiotic, (b) secreted by live bacteria as a product or byproduct of a metabolic process, and/or (c) released from a bacterial cell when the bacterial cell is lysed.

As used herein, unless otherwise specified, the term “therapeutically effective amount” refers to the amount of a substance that, when administered to a subject for treating a disease, or at least one of the clinical symptoms of a disease, is sufficient to effect such treatment of the disease or symptom thereof. The “therapeutically effective amount” may vary depending, for example, on the substance, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

As used herein, unless otherwise specified, the term “therapeutically effective dose” refers to a dose that provides effective treatment of a disease or disorder in a patient. A therapeutically effective dose may vary from substance to substance, and from patient to patient, and may depend upon factors such as the condition of the patient and the route of delivery. A therapeutically effective dose may be determined in accordance with routine pharmacological procedures known to those skilled in the art.

As used herein, the terms “treating” and “treatment” refer to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease.

The present disclosure provides postbiotic compositions that improve function, ameliorate dysfunction, and/or promote the resiliency of intra-and/or extravaginal tissues (e.g., the epithelial barrier, mucosal barrier, immunological milieu, and/or associated vaginal and/or dermatological microbiome) of the female genitourinary system. The disclosed postbiotic compositions include at least two components: (1) a paraprobiotic component comprising inactivated Lactobacillus crispatus cells (or one or more fractions, fragments, or lysates thereof), and (2) a metabolite component comprising a biologically significant amount (at least 5 milligrams per gram of the postbiotic composition) of D-lactate, which may (but need not) be naturally produced by the L. crispatus cells during fermentation. Without wishing to be bound by any particular theory, the present inventor hypothesizes that the postbiotic composition improves the intra-and extravaginal health of subjects to whom the composition is administered by any one or more of several mechanisms selected from enhancement of epithelial barrier integrity, inducing an immunological shift toward anti-inflammatory states, enhanced mucosal barrier function, pH reduction, and or pH-independent microbiome modulation.

One advantage and benefit of postbiotic compositions according to the present disclosure is that these compositions may have biological effects similar to probiotic (i.e., live-cell) compositions comprising the same or similar bacterial species or strains, but may have substantially improved shelf stability and/or improved consistency and/or reproducibility of effect and time to effect across diverse patient populations and disease states. Additionally, postbiotic compositions may have utility in applications where it is infeasible or impossible to stabilize a probiotic product, such as, for example, applications in which it is desirable to provide a therapeutic composition in an aqueous solution or suspension. Postbiotic compositions according to the present disclosure may have still further advantages relative to probiotic compositions in terms of suitability for administration to pediatric populations and/or severely ill or immunocompromised patients.

### Paraprobiotic Component and Microbial Strains Thereof, and Optional Probiotic Component

In general, it is not necessary that the inactivated Lactobacillus crispatus cells present in the paraprobiotic component of postbiotic compositions according to the present disclosure belong to any identified strain of L. crispatus; in other words, any inactivated L. crispatus cells may be used, without regard to specific strains, genomic characteristics, etc. In some embodiments, however, the postbiotic composition may further include a probiotic component, i.e., live bacterial cells, which may include live cells of L. crispatus. In some such embodiments, it may be desirable or preferable for the live bacterial cells of the probiotic component to be selected from certain specific strains or consortia of strains of L. crispatus, and where this is the case, it may also be desirable or preferable (based on cost or case of manufacture concerns, etc.) for the inactivated L. crispatus cells of the paraprobiotic component to belong to the same strains or consortia of strains as the live cells of the probiotic component. It is nonetheless to be expressly understood that the postbiotic compositions need not always include a probiotic component; that where a probiotic component is included, it need not always include L. crispatus; and that where a probiotic component comprising L. crispatus is included, the live L. crispatus cells of the probiotic component and the inactivated L. crispatus cells of the paraprobiotic component need not always be selected from the same strains or consortia of strains. In some embodiments of the postbiotic compositions of the present disclosure, the paraprobiotic component may include inactivated cells, and/or a probiotic component may include live cells, of one or more strains of L. crispatus selected from the group consisting of LUCA111 (ATCC Accession Deposit No. PTA-127219), LUCA011 (ATCC Accession Deposit No. PTA-127214), LUCA015 (ATCC Accession Deposit No. PTA-127215), LUCA009 (ATCC Accession Deposit No. PTA-127213), LUCA102 (ATCC Accession Deposit No. PTA-127217), LUCA006 (ATCC Accession Deposit No. PTA-127211), LUCA059 (ATCC Accession Deposit No. PTA-127216), LUCA103 (ATCC Accession Deposit No. PTA-127218), and LUCA008 (ATCC Accession Deposit No. PTA-127212).

The inactivated L. crispatus cells of the paraprobiotic component may be in any form, such as, by way of non-limiting example, a cell membrane-intact preparation of inactivated L. crispatus cells, a lysate preparation of inactivated L. crispatus cells, and/or a supernatant-enhanced preparation of inactivated L. crispatus cells. These and other preparations of inactivated L. crispatus cells may be produced by any suitable techniques known in the art, including but not limited to heat inactivation, ultraviolet inactivation, chemical treatment (e.g., treatment with formalin), gamma irradiation, sonication, tabletization, and the like. In some embodiments, the preparation of inactivated L. crispatus cells may be produced by a relatively mild heat treatment, e.g., heating a collection of live L. crispatus cells to a temperature of about 55° C. to about 125° C., or about 60° C. to about 120° C., or about 65° C. to about 115° C., or about 70° C. to about 110° C., or about 75° C. to about 105° C., or about 80° C. to about 100° C., or about 85° C. to about 95° C., or about 90° C. for a period of about 10 minutes to about 4 hours (or any value in any range having a lower bound of any whole number of minutes from 10 minutes to 240 minutes and an upper bound of any other whole number of minutes from 10 minutes to 240 minutes).

Those of ordinary skill in the art will readily appreciate that, where the postbiotic composition includes a probiotic component, it is inevitable that processing of the live bacterial cells into the postbiotic composition will result in death, inactivation, nonviability, etc. of some of the live bacterial cells, and that further cells will die, become inactive, become nonviable, etc. over time during the shelf life of the postbiotic composition. Thus, in some embodiments, those of ordinary skill in the art may take into account the relative quantities of live and inactivated bacterial cells provided at the outset of the manufacturing process, the type and parameters of the inactivation process, and other similar considerations to provide a postbiotic composition characterized by a predetermined ratio of inactivated bacterial cells to live bacterial cells (whether immediately upon formation of the postbiotic composition or a selected period of time thereafter). In some embodiments, the ratio of inactivated bacterial cells to live bacterial cells may be at least about 1:20, at least about 1:19, at least about 1:18, at least about 1:17, at least about 1:16, at least about 1:15, at least about 1:14, at least about 1:13, at least about 1:12, at least about 1:11, at least about 1:10, at least about 1:9, at least about 1:8, at least about 1:7, at least about 1:6, at least about 1:5, at least about 1:4, at least about 1:3, at least about 1:2, at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, or at least about 20:1, or alternatively in any range bounded by any two of these values.

### Metabolite Component

The metabolite component of postbiotic compositions according to the present disclosure provides D-lactate in an amount of at least about 5 mg/g (about 0.5 wt %), and most commonly about 5 mg/g to about 20 mg/g (about 0.5 wt % to about 2 wt %), of the overall postbiotic composition. The metabolite composition may optionally include other metabolites or postbiotic compounds in addition to D-lactate.

In many embodiments, it may be desirable or preferable for manufacture of the postbiotic composition to comprise culturing L. crispatus cells in a fermentation medium such that the L. crispatus cells produce D-lactate as a metabolic product or byproduct, inactivating the cultured L. crispatus cells, and formulating the inactivated L. crispatus cells and metabolically produced D-lactate (or portions thereof) into the postbiotic composition. In some embodiments, the D-lactate produced during fermentation by the L. crispatus cells may be all or substantially all of the lactate provided in the metabolite component of the postbiotic composition, but in other embodiments, exogenous sources of lactate (e.g., lactate salts, lactic acid, alkyl lactates, etc.) may be added to augment the “natural” D-lactate content produced by L. crispatus during fermentation; by way of non-limiting example, the amount of added lactate may be about 50% to about 250% of the amount of D-lactate produced by fermentation of L. crispatus (i.e., a ratio of “natural” D-lactate to added lactate may be from about 2:5 to about 2:1).

### Postbiotic Composition Formulations

In some embodiments, the postbiotic composition is formulated for intravaginal administration. Non-limiting examples of dosage forms into which the postbiotic compositions may suitably be formulated in these embodiments include a douche, a gel, an intrauterine device, an ointment, a vaginal ring, and a vaginal suppository (e.g., a capsule, a tablet, etc.). In certain embodiments of this type, a subject may apply the postbiotic composition intravaginally using an applicator, while in other embodiments the subject may apply the postbiotic composition into the vagina via digital insertion (i.e., using only the fingers). Intravaginal administration of such formulations may result in release and/or uptake of the synbiotic composition throughout the vaginal environment. Such intravaginal formulations may be prepared in a manner known in the pharmaceutical arts and comprise the postbiotic composition and at least one pharmaceutically acceptable vehicle. Intravaginal formulations may include a therapeutically effective amount of the postbiotic composition and a suitable amount of a pharmaceutically acceptable vehicle, so as to provide an appropriate form for administration to a patient. Pharmaceutically acceptable vehicles suitable for use in intravaginal formulations according to the present disclosure will be well known to those of ordinary skill in the art, but non-limiting examples of such vehicles include oil-in-water (O/W) emulsions, water-in-oil (W/O) emulsions, aqueous gels, alcohol-based gels, hydrocarbon bases (e.g., petrolatum), absorption bases, water-removable bases, water-soluble bases, aerosol or non-aerosol foams, aqueous or alcoholic solvents (or a combination thereof), aqueous suspension dispersion media, non-aqueous dispersion media, zinc oxide-based pastes, starch-based pastes, talcum powders, medicated powders, liposomes, niosomes, microemulsions, nanoemulsions, solid lipid nanoparticles, nanostructured lipid carriers, and carriers/excipients known in the art as being suitable for transdermal patches, matrix systems, reservoir systems, acrosol sprays, and pump sprays.

In some embodiments, the postbiotic composition is formulated for topical administration, and particularly for administration to any one or more external surfaces of the vulva (e.g., the labia majora, the labia minora, the vulvar vestibule, etc.). Non-limiting examples of dosage forms into which the postbiotic compositions may suitably be formulated in these embodiments include a balm, a cream, a dermal patch, a gel, a film, a hydrogel, an iontophoretic formulation, a liniment, a liposomal formulation, a lotion, an ointment, a paste, a shampoo (e.g., for application to the pubic hair and underlying skin), a solution, a transdermal patch, and a transdermal spray. Topical administration of such formulations may result in release and/or uptake of the synbiotic composition through the skin and/or mucosal surfaces to which the formulation is applied. Such topical formulations may be prepared in a manner known in the pharmaceutical arts and comprise the postbiotic composition and at least one pharmaceutically acceptable vehicle. Topical formulations may include a therapeutically effective amount of the postbiotic composition and a suitable amount of a pharmaceutically acceptable vehicle, so as to provide an appropriate form for administration to a patient. Pharmaceutically acceptable vehicles suitable for use in topical formulations according to the present disclosure will be well known to those of ordinary skill in the art, but non-limiting examples of such vehicles include oil-in-water (O/W) emulsions, water-in-oil (W/O) emulsions, aqueous gels, alcohol-based gels, hydrocarbon bases (e.g., petrolatum), absorption bases, water-removable bases, water-soluble bases, aerosol or non-aerosol foams, aqueous or alcoholic solvents (or a combination thereof), aqueous suspension dispersion media, non-aqueous dispersion media, zinc oxide-based pastes, starch-based pastes, talcum powders, medicated powders, liposomes, niosomes, microemulsions, nanoemulsions, solid lipid nanoparticles, nanostructured lipid carriers, and carriers/excipients known in the art as being suitable for transdermal patches, matrix systems, reservoir systems, aerosol sprays, and pump sprays.

Formulations of the postbiotic composition may be manufactured by means of conventional mixing, dissolving, granulating, dragee-making, levigating, emulsifying, encapsulating, entrapping, or lyophilizing processes. The formulations may be formulated in a conventional manner using one or more physiologically acceptable carriers, diluents, excipients, or auxiliaries, which facilitate processing of the postbiotic composition and one or more pharmaceutically acceptable vehicles into formulations that can be used pharmaceutically and/or therapeutically. Proper formulation is dependent upon the route of administration chosen. Formulations according to the present disclosure may take the form of sustained-release formulations suitable for administration to a patient.

Formulations according to the present disclosure may be formulated in a unit dosage form. A unit dosage form refers to a physically discrete unit suitable as a unitary dose for patients undergoing treatment, with each unit containing a predetermined quantity of the postbiotic composition calculated to produce an intended therapeutic effect. A unit dosage form may be for a single daily dose or for one of multiple daily doses, e.g., two times per day, three times per day, or more than three times per day. When multiple daily doses are used, a unit dosage form may be the same or different for each dose. One or more dosage forms may comprise a dose, which may be administered to a patient at a single point in time or during a time interval.

In certain embodiments, a dosage form provided by the present disclosure may be a controlled release dosage form. Controlled delivery technologies can improve the absorption and/or uptake of an active ingredient in a particular region or regions of the vaginal and/or vulvar environment. Controlled active ingredient delivery systems may be designed to deliver an active ingredient in such a way that the level of the active ingredient is maintained within a therapeutically effective window and effective and safe of the active ingredient in the vaginal and/or vulvar environment are maintained for a period as long as the system continues to deliver the active ingredient with a particular release profile. Controlled active ingredient delivery may produce substantially constant levels of an active ingredient in the vaginal and/or vulvar environment over a period of time as compared to fluctuations observed with immediate release dosage forms. For some applications, maintaining a constant blood and/or tissue concentration throughout the course of therapy is the most desirable mode of treatment. Immediate release of an active ingredient may cause levels of one or more active ingredients (e.g., the paraprobiotic component and/or D-lactate) to peak above the level required to elicit a desired response, which may waste the active ingredient(s) and may cause or exacerbate toxic side effects. Controlled active ingredient delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, and the like.

An appropriate dosage form for a particular formulation provided by the present disclosure may depend, at least in part, on the intravaginal and/or topical absorption properties of the active ingredient(s) and/or the stability of the active ingredient(s) in the vaginal and/or vulvar environments, the pharmacokinetics of the active ingredient(s), and the intended therapeutic profile. An appropriate controlled release dosage form may be selected for a particular ingredient or combination of ingredients.

In certain embodiments, formulations according to the present disclosure may be provided in dosage forms adapted to provide sustained release of the postbiotic composition upon intravaginal and/or topical administration. Sustained release dosage forms may be used to release active ingredients over a prolonged time period. Sustained release dosage forms include any dosage form that maintains therapeutic concentrations of an active ingredient in a biological fluid such as the plasma, blood, cerebrospinal fluid, or in a tissue or organ for a prolonged time period.

Sustained release dosage forms include diffusion-controlled systems such as reservoir devices and matrix devices, dissolution-controlled systems, osmotic systems, and erosion-controlled systems. Sustained release dosage forms and methods of preparing the same are well known in the art.

In one embodiment, an intravaginal dosage form is administered to a patient at a dosing frequency of three times per day. In another embodiment, the intravaginal dosage form is administered to a patient at a dosing frequency of twice per day. In still another embodiment, the intravaginal dosage form is administered to a patient at a dosing frequency of once per day.

In one embodiment, the topical dosage form is administered to a patient at a dosing frequency of three times per day. In another embodiment, the topical dosage form is administered to a patient at a dosing frequency of twice per day. In still another embodiment, the topical dosage form is administered to a patient at a dosing frequency of once per day.

In certain embodiments, formulations according to the present disclosure may include any suitable dosage forms that achieve the above described in vitro and/or in vivo release profiles. Examples of suitable dosage forms are described herein. Those skilled in the formulation art can develop any number of acceptable dosage forms given the dosage forms described in the examples as a starting point.

An appropriate dose of the postbiotic composition may be determined according to any one of several well-established protocols. For example, animal studies, such as studies using mice, rats, dogs, and/or monkeys, may be used to determine an appropriate dose of the postbiotic composition. Results from animal studies may be extrapolated to determine doses for use in other species, such as, for example, humans.

In some embodiments, particularly (but by no means exclusively) those embodiments in which the postbiotic composition includes a probiotic component, formulations according to the present disclosure may further include a prebiotic component. The prebiotic component may include any one or more nutrients (e.g., monosaccharides, disaccharides, oligosaccharides, polysaccharides, lipids, amino acids, peptides, proteins, etc.). In some embodiments, it may be particularly preferable to provide a prebiotic composition comprising maltose.

In some embodiments, formulations according to the present disclosure may further include one or more redox potential control agents. Non-limiting examples of suitable redox potential control agents include cysteine, glutathione, selenium, N-acetylcysteine, vitamin C, vitamin E, alpha-lipoic acid, CoQ10, flavonoids, thioredexin, and combinations thereof.

In some embodiments, formulations according to the present disclosure may further include one or more pH buffers configured to maintain a healthy pH of the vaginal environment. Non-limiting examples of suitable pH buffers include citrates (e.g., magnesium citrate), carbonates (e.g., calcium bicarbonate), phosphates (e.g., sodium phosphate), lactates (e.g., calcium lactate), and combinations thereof.

In some embodiments, formulations according to the present disclosure may further include one or more antimicrobial agents. Non-limiting examples of suitable antimicrobial agents include antibiotics (e.g., penicillins, cephalosporins, macrolides, tetracyclines, aminoglycosides, fluoroquinolones, sulfonamides), antifungals (e.g., azoles, echinocandins, polyenes), antivirals (e.g., nucleoside analogs, protease inhibitors, neuraminidase inhibitors, polymerase inhibitors), antiparasitics (e.g., antimalarials, antiprotozoals, antihelminthics), antiseptics and disinfectants (e.g., alcohols, chlorhexidine, hydrogen peroxide, iodine, quaternary ammonium compounds, and other pharmaceutically acceptable surfactants), neem, oregano, cinnamon, ginger, turmeric, bacteriocins, lysozymes, bee propolis, colloidal silver, apple cider vinegar, and combinations thereof.

In some embodiments, formulations according to the present disclosure may further include one or more growth promoters. Non-limiting examples of suitable growth promoters include maltose, lactulose, fructooligosaccharides, inulin, galactooligosaccharides, alpha-glucans, beta-glucans, mannose, glycerin, arabinogalactan, xylooligosaccharides, and combinations thereof.

It is to be expressly understood that formulations according to the present disclosure may further include compounds, materials, substances, etc. other than inactivated Lactobacillus crispatus cells and D-lactate that exert advantageous and/or beneficial therapeutic and/or cosmetic effects when administered to a subject intravaginally and/or topically. A first non-limiting example of such a substance is niacinamide, which may help protect the subject against acne and/or one or more types of skin cancer when administered topically. A second non-limiting example of such a substance is lactoferrin, which may provide an antibacterial, antiviral, antifungal, and/or anticarcinogenic effect to the subject. A third non-limiting example of such a substance is squalane, which may provide an emollient effect to the skin of the subject when administered topically. A fourth non-limiting example of such a substance is hyaluronic acid, which may improve wound healing and/or skin barrier function in the subject when administered topically.

### Uses

The methods and formulations disclosed herein can be used to treat patients suffering from diseases, disorders, conditions, and symptoms for which postbiotic compositions are known to provide or are later found to provide therapeutic benefit. Formulations disclosed herein can be used to treat, prevent, or reduce the likelihood of a disease, disorder, condition, or symptom chosen from, by way of non-limiting example, bacterial vaginosis, pelvic inflammatory disease, sexually transmitted infection, postpartum endometritis and/or sepsis, preterm birth, preterm premature rupture of membranes, miscarriage, chorioamnionitis and/or intra-amniotic infection, infertility and/or ineffectiveness of infertility treatment, cervical intraepithelial neoplasia, cervical cancer, and/or another cancer of the female genitourinary system.

Methods of treating a disease in a patient provided by the present disclosure comprise administering to a patient in need of such treatment a therapeutically effective amount of a postbiotic composition of the disclosure. These methods and formulations provide therapeutic or prophylactic amounts of the D-lactate (and, if included, other compounds) of the metabolite component and/or of the inactivated Lactobacillus crispatus cells of the paraprobiotic component following administration to a patient. The postbiotic composition may be administered in an amount and using a dosing schedule as appropriate for treatment of a particular disease.

In embodiments of the present disclosure, daily doses of D-lactate in the metabolite component of the postbiotic composition may be about 0.5 mg to about 25 mg, or alternatively any value in any range having a lower bound of any whole number of tenths of milligrams from 0.5 mg to 25 mg and an upper bound of any other whole number of tenths of milligrams from 0.5 mg to 25 mg; in particular embodiments, the daily dose may be about 6 mg to about 20 mg, or about 12 mg. Unit dosage forms according to the present disclosure may be formulated so as to provide the entire dose in a single dosage form intended for once-daily administration, or may be formulated so as to divide this daily dose (equally or unequally) between each of several dosage forms intended for administration multiple times per day; for example, where the postbiotic composition is formulated so as to provide a daily dose of 9 mg D-lactate per day, unit dosage forms may be formulated as a 9 mg dosage form intended to be administered once per day, a 4.5 mg dosage form intended to be administered twice per day, a 3 mg dosage form intended to be administered three times per day, and so on.

In certain embodiments, microbial strains of the paraprobiotic component may be administered at a dose over time from about 1 billion inactivated cells to about 100 billion inactivated cells per day, or alternatively any value in any range having a lower bound of any number of hundreds of millions of inactivated cells per day from 1 billion to 100 billion and an upper bound of any other number of hundreds of millions of inactivated cells per day from 1 billion to 100 billion.

An appropriate dose of postbiotic composition may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

The postbiotic composition may be assayed in vitro and in vivo for the desired therapeutic or prophylactic activity prior to use in humans. In vivo assays, for example using appropriate animal models, may also be used to determine whether administration of postbiotic composition is therapeutically effective.

In certain embodiments, a therapeutically effective dose of the postbiotic composition may provide therapeutic benefit without causing substantial toxicity including adverse side effects. Toxicity of the postbiotic composition and/or metabolites thereof may be determined using standard pharmaceutical procedures and may be ascertained by those skilled in the art. The dose ratio between toxic and therapeutic effect is the therapeutic index. A dose of the postbiotic composition may be within a range capable of establishing and maintaining a therapeutically effective concentration of D-lactate and/or inactivated L. crispatus cells in the vaginal and/or vulvar environment.

Postbiotic composition administration may be used to treat a disease, disorder, condition, or symptom chosen from, by way of non-limiting example, bacterial vaginosis, pelvic inflammatory disease, sexually transmitted infection, postpartum endometritis and/or sepsis, preterm birth, preterm premature rupture of membranes, miscarriage, chorioamnionitis and/or intra-amniotic infection, infertility and/or ineffectiveness of infertility treatment, cervical intraepithelial neoplasia, cervical cancer, and/or another cancer of the female genitourinary system. The underlying etiology of any of the foregoing diseases being treated may have a multiplicity of origins. Further, in certain embodiments, a therapeutically effective amount of postbiotic composition may be administered to a patient, such as a human, as a preventative measure against, and/or a measure that reduces the likelihood of, the foregoing diseases and disorders. Thus, a therapeutically effective amount of postbiotic composition may be administered as a preventative and/or prophylactic measure to a patient having a predisposition for and/or history of a disease, disorder, condition, or symptom chosen from, by way of non-limiting example, bacterial vaginosis, pelvic inflammatory disease, sexually transmitted infection, postpartum endometritis and/or sepsis, preterm birth, preterm premature rupture of membranes, miscarriage, chorioamnionitis and/or intra-amniotic infection, infertility and/or ineffectiveness of infertility treatment, cervical intraepithelial neoplasia, cervical cancer, and/or another cancer of the female genitourinary system.

The disclosure is further described by reference to the following non-limiting Examples. The Examples illustrate various aspects of the disclosure. It will be apparent to those skilled in the art that many modifications, to both materials and methods, may be practiced without departing from the scope of the disclosure.

### Example 1

### Lactobacillus Crispatus Fermentation

A culture medium was prepared by dissolving solid medium components in water at the concentrations shown in Table 1 below. This medium was autoclaved at 121° C. for 20 minutes and then supplemented with a filtered dextrose solution, which was added in an amount sufficient to achieve a dextrose concentration in the culture medium of 70.0 g/L.

TABLE 1

| Component | Concentration (g/L) |
|---|---|
| Yeast extract | 10.0 |
| Pea peptone | 40.0 |
| Potassium phosphate dibasic | 4.0 |
| Sodium acetate | 10.0 |
| Ammonium citrate | 4.0 |
| Magnesium sulfate | 0.4 |
| Manganese sulfate | 0.1 |
| Polysorbate 80 | 2.0 |

This medium was then used to prepare three seed cultures; particularly, cryovials of master cell banks (MCBs) of one of three strains of Lactobacillus crispatus, namely strains LUCA011 (ATCC Accession Deposit No. PTA-127214), LUCA009 (ATCC Accession Deposit No. PTA-127213), and LUCA103 (ATCC Accession Deposit No. PTA-127218), were thawed and used to inoculate separate aliquots of medium at an inoculum percentage of 0.05%. The volume of each aliquot of seed culture was 17.5 L. The seed culture was incubated at 37±2° C. at an initial pH of 5.7±0.2. The seed tank was overlaid with compressed air in aerobic conditions and agitated at 240 rpm. The culture time was approximately 22-24 hours. The optical density (OD) of the seed culture medium was monitored from t=15 hours onward. Cultures were stopped either when the optical density exceeded 7 or if the growth of L. crispatus was observed to slow.

The main fermentation culture medium was adjusted to a pH of 5.7±0.2. Each seed culture was used to inoculate a separate bioreactor at an inoculation percentage of 0.5%. The fermentation culture was incubated at 37±2° C. in aerobic conditions with a pH setpoint of 5.5. The agitator speed was maintained at 130 rpm. The OD and glucose concentration of the main fermentation culture medium were monitored from t=10 hours onward. The culture time was approximately 15-18 hours. Cultures were stopped either when the glucose concentration fell below 5 g/L or if the growth of L. crispatus was observed to slow. A viable cell count was measured at the end of each culture; these counts were 4.7 billion colony forming units (CFU) for strain LUCA009, 1.20 billion CFU for strain LUCA011, and 890 million CFU for strain LUCA103.

### Example 2

### D-lactate Production of Lactobacillus Crispatus

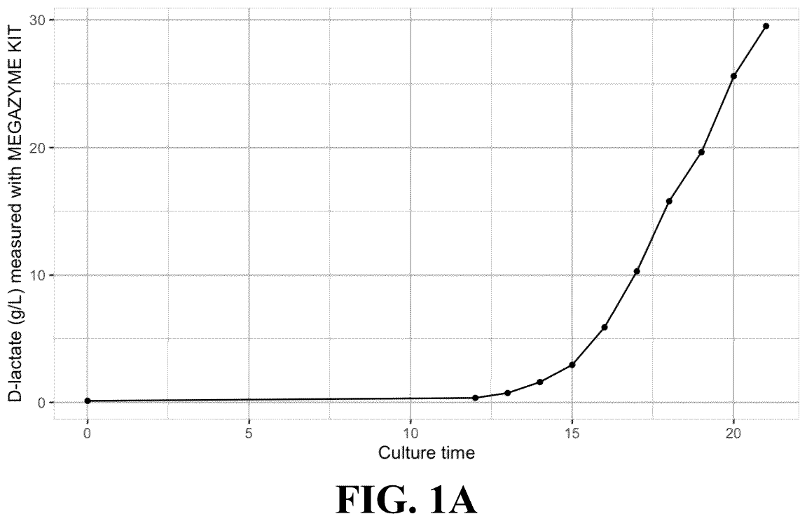

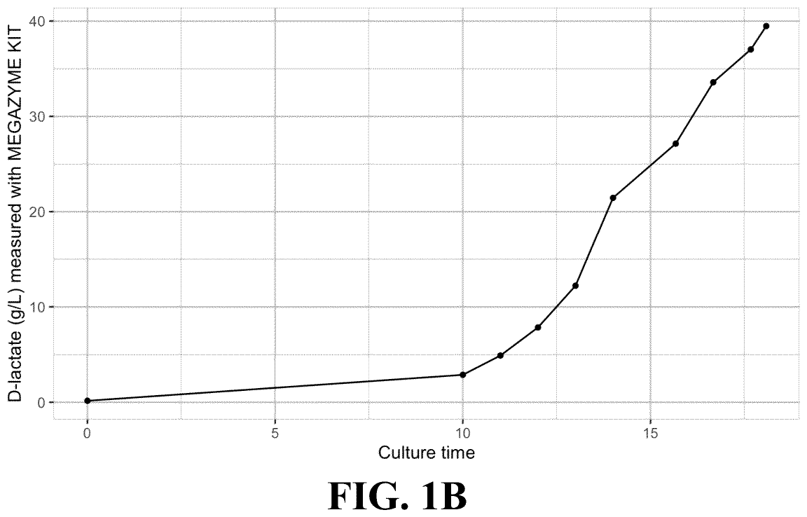

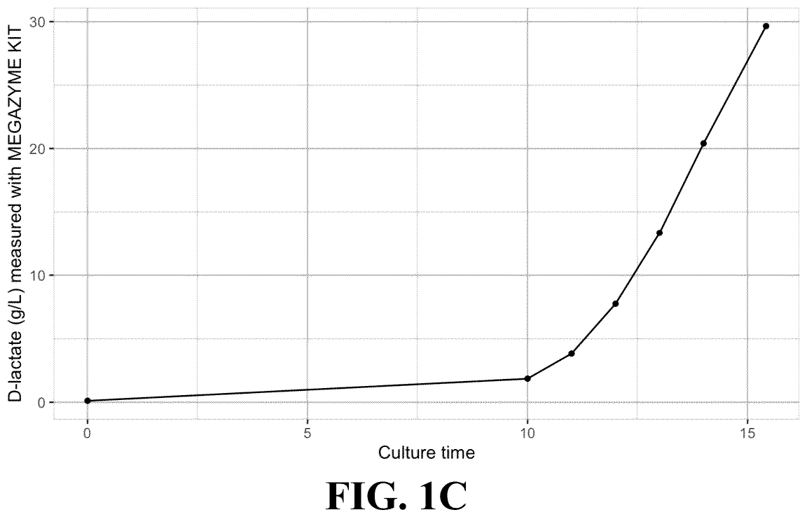

At t=12 hours and each hour thereafter during the fermentations described in Example 1, the D-lactate concentration in each main fermentation culture was measured. FIGS. 1A, 1B, and 1C are graphs of this concentration with respect to LUCA009 fermentation, LUCA011 fermentation, and LUCA103 fermentation, respectively (the units of time on the x-axis of each graph are hours). As FIGS. 1A through 1C illustrate, the final D-lactate concentration in each culture ranged from about 25 to about 40 g/L.

### Example 3

### Preparation of Postbiotic Tablets Containing Inactivated Lactobacillus Crispatus and D-lactate

Each of the three fermentation broths described in Examples 1 and 2 was harvested at the end of fermentation and centrifuged to separate a cell paste (precipitate) from the spent culture medium (supernatant). The cell paste was tabletized, which resulted in the inactivation of a significant proportion of the L. crispatus cells, and the supernatant was discarded; while a portion of the D-lactate produced by the L. crispatus fermentation was discarded as part of the supernatant, a significant amount of D-lactate remained in the cell paste.

Tabletization of the cell paste into 850 mg tablets suitable for administration to a subject was carried out via a conventional tabletization method (compression with cellulose, hydroxypropyl methylcellulose, and magnesium stearate) and the tablets were then held under ambient conditions for nine months. After nine months, the D-lactate content of each of three tablets was assessed. The results are shown in Table 2.

TABLE 2

D-lactate content

| Sample | wt % of tablet | mg/tablet |
|---|---|---|
| 1 | 1.28 | 10.88 |
| 2 | 1.43 | 12.16 |
| 3 | 1.47 | 12.50 |

### Example 4

### Improvement of Barrier Function by Application of Postbiotic L. Crispatus/D-lactate Compositions

EpiVaginal (VEC-100) 3-D tissues, produced by MatTek Corporation, were used to evaluate eight test conditions plus a positive control known to have negative effects on tissue viability and barrier function (nonoxynol-9); the conditions tested are given in Table 3 below. Barrier integrity was measured at baseline, and tissues were treated in triplicate with test articles for 24 hours. Three tissues were left untreated to serve as negative control (NC); three tissues were treated with 4% nonoxynol-9 to serve as positive control (PC); and three tissues were dosed with media to serve as vehicle control (VC). At the end of 24 hr exposure to the test articles, barrier integrity was measured using transepithelial electrical resistance (TEER) and two tissues were used to assess tissue viability with the MTT assay. From each treatment group, one tissue was fixed in 10% formalin and processed for H&E staining for histological analysis.

Throughout this Example and Example 8 below, “VS-01” refers to a consortium of three Lactobacillus crispatus strains: LUCA011 (ATCC Accession Deposit No. PTA-127214) at a concentration of 3.70·10¹⁰ CFU/g, LUCA009 (ATCC Accession Deposit No. PTA-127213) at a concentration of 8.60·10⁹ CFU/g, and LUCA103 (ATCC Accession Deposit No. PTA-127218) at a concentration of 2.50·10¹⁰ CFU/g. A stock of 10⁶ CFU/mL of the combined strains was used for testing. A comparator strain, L. crispatus ATCC 33197, was sourced from the American Type Culture Collection, and it was determined by bioinformatic analysis that the genome of this strain had between 99.0% and 99.2% average nucleotide identity (ANI) with each of the three VS-01 strains.

TABLE 3

| Condition ID | Description |
|---|---|
| PC1 | 4% nonoxynol-9 |
| NC1 | No treatment |
| VC | Cell medium (VEC-100-AFAB, lactate-free) |
| TA1 | VS-01, live, 10⁶ CFU |
| TA2 | VS-01, heat-killed, 10⁶ CFU |
| TA3 | VS-01, heat-killed, 10⁵ CFU |
| TA4 | 100 mM D-lactate + VS-01, heat-killed, 10⁶ CFU |
| TA5 | 10 mM D-lactate + VS-01, heat-killed, 10⁶ CFU |
| TA6 | 10 mM D-lactate |
| TA7 | 100 mM D-Lactate |
| TA8 | L. crispatus ATCC 33197, heat-killed, 10⁶ CFU |

The concentration of VS-01 most relevant to in vivo exposure in the VEC-100 model (10⁶ CFU/mL) was determined using two different approaches: (1) Comparison of the exposure per unit area of vaginal surface area between VS-01 in vivo and VS-01 in the VEC-100 model. As known from the literature, the average surface area of the human vagina ranges from 65.73 to 107.07 cm² with a mean of 87.46 cm² and a standard deviation of 7.80 cm². If an 850 mg tablet as described in Example 3 instantaneously dissolved, the estimated coverage would be 9.7 mg/cm² (850 mg/87.46 cm²). This most closely corresponds to a 1:100 dilution of the tablet and can be considered an upper bound, given that the tablet dissolves slowly over a 24-hour period. (2)

Comparison of the ratio of L. crispatus cells to vaginal epithelial cells in vivo and in the VEC-100 model. The Mattek model does not include a “native microbiome” aspect, but the literature indicates that the native vaginal microbiome is present at about a 10:1 bacterial cell/epithelial cell ratio. The Mattek model contains between about 350,000 and about 500,000 cells at a depth of three to five layers. If 100,000 epithelial cells are exposed at the surface, exposure to 10⁶ bacterial cells produces a reasonable estimate of a 10:1 bacterial cell/epithelial cell ratio.

An estimate of the concentration of D-lactate most relevant to in vivo exposure was obtained considering tablet dissolution kinetics. Tablets as described in Example 3 are slow-release formulations, dissolving over the course of about twelve hours. The volume of cervicovaginal fluid (CVF) present in the vagina is variable, ranging from about 1 mL to about 4 mL, with a discharge rate of about 2 mL to about 5 mL over 24 hours; thus, the dissolution rate of a tablet as described in Example 3 would be about 71 mg/hour. D-lactate concentrations of tablets as described in Example 3 range from about 34 mg/tablet to about 44 mg/tablet. Estimating that 8.4% of an 850 mg tablet dissolves per hour (71 mg/hour divided by 850 mg/tablet) gives an estimated in vivo D-lactate concentration range of 31.7 mM to 40.44 mM D-lactate. Therefore, testing 10 mM and 100 mM D-lactate in this Example covers the physiological range of estimated exposure given the expected variability in the exact concentration of D-lactate per tablet.

Results of this testing are given in Table 4; the reported TEER values are normalized to the average TEER value obtained for test condition VC (i.e., TEER values reported in Table 4 of less than 100% indicate a decrease in TEER relative to the mean for test condition VC, and TEER values reported in Table 4 of more than 100% indicate an increase in TEER relative to the mean for test condition VC).

TABLE 4

| | | TEER | T-test | T-test | T-test | T-test |
|---|---|---|---|---|---|---|
| Condition | TEER avg. | std. dev. | vs. | vs. | vs. | vs. |
| ID | (% of VC) | (% of VC) | VC | TA2 | TA8 | TA1 |
| VC | 100.0 | 14.1 | 1.000 | — | — | — |
| PC | 0.6 | 0.5 | 0.007 | — | — | — |
| TA1 | 163.1 | 6.4 | 0.007 | 0.417 | 0.056 | — |
| TA2 | 186.1 | 39.2 | 0.050 | 1.000 | 0.053 | — |
| TA3 | 121.8 | 20.5 | 0.213 | 0.086 | 0.505 | — |
| TA4 | 138.8 | 49.7 | 0.308 | 0.268 | 0.410 | 0.486 |
| TA5 | 128.6 | 41.5 | 0.358 | 0.156 | 0.512 | 0.286 |
| TA6 | 128.4 | 20.4 | 0.127 | 0.109 | 0.341 | 0.087 |
| TA7 | 114.8 | 14.2 | 0.268 | 0.074 | 0.709 | 0.015 |
| TA8 | 108.0 | 25.3 | 0.666 | 0.053 | 1.000 | — |

All eight test articles were non-cytotoxic, preserved barrier function, and showed histological integrity after 24-hour exposure. As Table 4 demonstrates, both the live cell (TA1) and heat-killed (TA2) postbiotic mixtures showed statistically significant improvement in barrier integrity, suggesting potential benefits for epithelial maintenance. The controls behaved as expected in the VEC-100 model, lending confidence to the results. Specifically, the positive control (nonoxynol-9) caused severe cytotoxicity and barrier disruption relative to vehicle and negative control treatments. As expected, the vehicle control (VC) cell medium treatment reduced both viability and barrier function relative to untreated negative control. Therefore, readouts from all test articles dissolved in media were compared to the vehicle control results.

As expected, the positive control showed near-total barrier disruption (0.2% vs NC, 0.6% vs VC). The VC alone reduced TEER to 31.4% vs NC. All test articles had higher TEER than VC, and TA1 and TA2 showed statistically significant improvement in TEER over VC. There was no statistically significant difference in TEER between live cells (TA1) and heat-killed cells (TA2) of the VS-01 consortium (p=0.417), indicating effectively equivalent performance in improving epithelial barrier between these two treatments. Notably, both TA1 and TA2 showed significant improvements (p=0.056 and p=0.053, respectively) in barrier integrity over the comparator strain of L. crispatus (TA8). TA2 showed a mean TEER improvement over VC of 186% compared to just 108% for the comparator strain.

The addition of D-lactate at 10 mM and 100 mM to heat-killed VS-01 cells did not result in significant improvements in TEER over heat-killed VS-01 cells alone. Additionally, D-lactate alone did not result in significant improvements in TEER over VC. Interestingly, heat-killed VS-01 cells trended towards higher levels of TEER than 100 mM D-lactate treatment (p=0.074), but not 10 mM D-lactate treatment (p=0.109). Live VS-01 cells showed significantly higher TEER than 100 mM D-lactate (0.015) but not 10 mM D-lactate (p=0.087).

This Example demonstrates at least the following four findings: (1) At equivalent concentrations, a postbiotic composition comprising heat-killed L. crispatus cells alone supports epithelial barrier integrity as well as live cells of the same L. crispatus strains (in this case, the VS-01 consortium strains). There was no significant difference in TEER improvement between the live cell and heat-killed cell treatments, and both treatments showed significant improvement in TEER over vehicle control. (2) At equivalent concentrations, a postbiotic composition comprising a combination of D-lactate and heat-killed L. crispatus cells supports epithelial barrier integrity as well as live cells of the same L. crispatus strains (in this case, the VS-01 consortium strains). There was no significant difference in TEER between the D-lactate+heat-killed cell treatments and the live cell treatment. (3) Regardless of concentration, or at least over a wide range of concentrations, heat-killed L. crispatus cell treatments show significant improvements in TEER over vehicle control, where D-lactate alone does not. (4) At equivalent concentrations, a postbiotic composition comprising heat-killed L. crispatus cells of the VS-01 consortium supports epithelial barrier integrity as well as, or better than, heat-killed cells of a comparator L. crispatus strain. The heat-killed VS-01 cell treatments showed significant (p=0.053) improvements in TEER compared to treatments with heat-killed cells of the comparator strain.

### Example 5

### Anti-inflammatory Effect of Postbiotic L. Crispatus/D-lactate Compositions

A postbiotic composition made substantially as described in Examples 1-3 and throughout this disclosure is introduced to a single layer or multilayer culture of vaginal epithelial cells (VECs) with or without agonist stimulation. Two types of negative control (a “negative control postbiotic material” created by lyophilizing a non-L. crispatus bacterial species and/or treating a non-L. crispatus bacterial species in the same way as the L. crispatus, and a supernatant of this bacterial species) are also introduced to similar single layer or multilayer VEC cultures. It is found that the postbiotic composition as disclosed herein reduces markers of inflammation in the VEC culture to a greater extent than the two negative controls.

### Example 6

### Inhibition of Vaginal Pathogen Infection by Postbiotic L. Crispatus/D-lactate Compositions

A postbiotic composition made substantially as described in Examples 1-3 and throughout this disclosure is introduced to a single or multilayer culture of VECs inoculated with a panel of known vaginal pathogens, including Candida albicans and Chlamydia trachomatis; the postbiotic composition is applied to the VEC culture either before or after inoculation of the culture with the pathogens (i.e., the VEC culture may or may not be “pre-incubated” with the postbiotic material prior to pathogen inoculation). Two types of negative control (a “negative control postbiotic material” created by lyophilizing a non-L. crispatus bacterial species and/or treating a non-L. crispatus bacterial species in the same way as the L. crispatus, and a supernatant of this bacterial species) are also introduced to similar single or multilayer VEC cultures. It is found that the postbiotic composition as disclosed herein reduces and/or prevents infection of the VECs or HeLa cells by the vaginal pathogens to a greater extent than the two negative controls.

### Example 7

### Inhibition of C. Albicans Adherence to Epithelial Cells

A postbiotic composition made substantially as described in Examples 1-3 and throughout this disclosure is introduced to a culture of VEC or Hela cells. A “negative control postbiotic material” created by lyophilizing a non-L. crispatus bacterial species and/or treating a non-L. crispatus bacterial species in the same way as the L. crispatus is also introduced to similar VEC or HeLa cultures. It is found that pre-incubation of VEC or HeLa cells with the postbiotic composition as disclosed herein reduces and/or prevents the adhesion of Candida albicans and/or other Candida spp. to VEC or HeLa cells to a greater extent than the negative control postbiotic material. Without wishing to be bound by any particular theory, the present inventor hypothesizes that this effect can be attributed to promotion, by the postbiotic composition of the present disclosure, of secretion of human beta-defensin-3 by the VEC or HeLa cells.

### Example 8

### Upregulation of Anti-inflammatory Cytokines by Postbiotic L. Crispatus/D-lactate Compositions

A postbiotic composition made substantially as described in Examples 1-3 and throughout this disclosure is introduced to a dendritic cell culture. Two types of negative control (a “negative control postbiotic material” created by lyophilizing a non-L. crispatus bacterial species and/or treating a non-L. crispatus bacterial species in the same way as the L. crispatus, and a supernatant of this bacterial species) are also introduced to similar dendritic cell cultures. The levels of anti-inflammatory cytokines in the dendritic cell cultures are measured. It is found that the postbiotic composition as disclosed herein upregulates production of the anti-inflammatory cytokines to a greater extent than the two negative controls.

### Example 9

### Anti-pathogenic Biosurfactant Activity of Postbiotic L. Crispatus/D-lactate Compositions

A postbiotic composition made substantially as described in Examples 1-3 and throughout this disclosure, and/or a resuspension of biosurfactants isolated from such a composition, is incubated in vitro in the presence of known vaginal pathogens. Two types of negative control (a “negative control postbiotic material” created by lyophilizing a non-L. crispatus bacterial species and/or treating a non-L. crispatus bacterial species in the same way as the L. crispatus, and a supernatant of this bacterial species) are similarly incubated. It is found that the postbiotic composition as disclosed herein and/or biosurfactants isolated therefrom have greater antimicrobial activity against the vaginal pathogens than the two negative controls.

### Example 10

### S-layer Protein Activity of Postbiotic L. Crispatus/D-lactate Compositions

A postbiotic composition made substantially as described in Examples 1-3 and throughout this disclosure, and/or a composition of S-layer proteins isolated from such a composition, is introduced to a multilayer culture of VECs with or without agonist stimulation. Two types of negative control (a “negative control postbiotic material” created by lyophilizing a non-L. crispatus bacterial species and/or treating a non-L. crispatus bacterial species in the same way as the L. crispatus, and a supernatant of this bacterial species) are also introduced to similar multilayer VEC cultures. It is found that the S-layer proteins of the postbiotic composition as disclosed herein adhere to VEC surfaces better than proteins present in the two negative controls.

The concepts illustratively disclosed herein suitably may be practiced in the absence of any clement which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

For purposes of further disclosure and to comply with applicable written description and enablement requirements, the following references are incorporated by reference in their entireties:

U.S. patent application Ser. Nos. 16/800,702, 16/917,661, 17/223,710, 17/223,754, 17/567,295, 18/045,066, 18/087,545, 18/130,946, 18/232,433, and 18/235,686; Australian Patent Application 2022202848; Canadian Patent Application 3157238; and European Patent Application 20877272.3.

Antonietta Rizzo, Antonio Losacco, Caterina Romano Carratelli, Lactobacillus crispatus modulates epithelial cell defense against Candida albicans through Toll-like receptors 2 and 4, interleukin 8 and human β-defensins 2 and 3, Immunology Letters, Volume 156, Issues 1-2, 2013, Pages 102-109, ISSN 0165-2478, https://doi.org/10.1016/j.imlet.2013.08.013.

Sharma D., Saharan B. S. Functional characterization of biomedical potential of biosurfactant produced by Lactobacillus helveticus. Biotechnol. Rep. (Amst) 2016;11:27-35. doi: 10.1016/j.btre.2016.05.001.

De Gregorio, P. R., Parolin, C., Abruzzo, A. et al. Biosurfactant from vaginal Lactobacillus crispatus BC1 as a promising agent to interfere with Candida adhesion. Microb Cell Fact 19, 133 (2020). https://doi.org/10.1186/s12934-020-01390-5.

Rose W A, McGowin C L, Spagnuolo R A, Eaves-Pyles T D, Popov V L, Pyles R B. Commensal Bacteria Modulate Innate Immune Responses of Vaginal Epithelial Cell Multilayer Cultures. PLOS ONE. 2012;7 (3): e32728. doi: 10.1371/journal.pone.0032728.

Eslami S, Hadjati J, Motevaseli E, et al. Lactobacillus crispatus strain SJ-3C-US induces human dendritic cells (DCs) maturation and confers an anti-inflammatory phenotype to DCs. APMIS. 2016;124 (8): 697-710. doi: 10.1111/apm.12556.

Joseph, A., Anton, L., Guan, Y. et al. Extracellular vesicles from vaginal Gardnerella vaginalis and Mobiluncus mulieris contain distinct proteomic cargo and induce inflammatory pathways. npj Biofilms Microbiomes 10, 28 (2024). https://doi.org/10.1038/s41522-024-00502-y.

Hynonen, U. & Palva, A. Lactobacillus surface layer proteins: structure, function and applications. Appl Microbiol Biotechnol. 97, 5225-5243 (2013).

Abramov, V. et al. Probiotic Properties of Lactobacillus crispatus 2,029: Homeostatic Interaction with Cervicovaginal Epithelial Cells and Antagonistic Activity to Genitourinary Pathogens. Probiotics Antimicrob. Proteins 6, 165-176 (2014).

Abramov, V. M. et al. S-layer protein 2 of Lactobacillus crispatus 2029, its structural and immunomodulatory characteristics and roles in protective potential of the whole bacteria against foodborne pathogens. Int J. Biol. Macromol. 150, 400-412 (2020).

Chen H, Min S, Wang L, Zhao L, Luo F, Lei W, Wen Y, Luo L, Zhou Q, Peng L, Li Z. Lactobacillus Modulates Chlamydia Infectivity and Genital Tract Pathology in vitro and in vivo. Front Microbiol. 2022 Apr. 28;13:877223. doi: 10.3389/fmich.2022.877223. PMID: 35572713; PMCID: PMC9098263.

Rizzo A. Losacco A. Carratelli C R. Lactobacillus crispatus modulates epithelial cell defense against Candida albicans through Toll-like receptors 2 and 4, interleukin 8 and human β-defensins 2 and 3. Immunol Lett. 2013; 156 (1-2): 102-109. doi: 10.1016/j.imlet.2013.08.013.

1. A postbiotic composition, comprising:
a paraprobiotic component, comprising inactivated cells of Lactobacillus crispatus; and
a metabolite component, comprising D-lactate in an amount of at least about 5 milligrams per gram of the postbiotic composition.

2. The postbiotic composition of claim 1, further comprising a probiotic component.

3. The postbiotic composition of claim 2, wherein the probiotic component comprises live cells of Lactobacillus crispatus.

4. The postbiotic composition of claim 3, wherein at least a portion of the inactivated cells of Lactobacillus crispatus in the paraprobiotic component are of the same strain or consortium of strains as at least a portion of the live cells of Lactobacillus crispatus in the probiotic component.

5. The postbiotic composition of claim 3, wherein the inactivated cells of Lactobacillus crispatus in the paraprobiotic component, the live cells of Lactobacillus crispatus in the probiotic component, or both comprise cells of at least one strain of Lactobacillus crispatus selected from the group consisting of LUCA111 (ATCC Accession Deposit No. PTA-127219), LUCA011 (ATCC Accession Deposit No. PTA-127214), LUCA015 (ATCC Accession Deposit No. PTA-127215), LUCA009 (ATCC Accession Deposit No. PTA-127213), LUCA102 (ATCC Accession Deposit No. PTA-127217), LUCA006 (ATCC Accession Deposit No. PTA-127211), LUCA059 (ATCC Accession Deposit No. PTA-127216), LUCA103 (ATCC Accession Deposit No. PTA-127218), and LUCA008 (ATCC Accession Deposit No. PTA-127212).

6. The postbiotic composition of claim 1, wherein at least a portion of the D-lactate in the metabolite component is produced as a product or byproduct of a metabolic process of live Lactobacillus crispatus cells.

7. The postbiotic composition of claim 6, wherein at least a portion of the live Lactobacillus crispatus cells that produce the at least a portion of the D-lactate in the metabolite component are subsequently inactivated and form at least a portion of the inactivated Lactobacillus crispatus cells of the paraprobiotic component.

8. The postbiotic composition of claim 6, wherein substantially all of the D-lactate in the metabolite component is produced as a product or byproduct of a metabolic process of live Lactobacillus crispatus cells.

9. The postbiotic composition of claim 6, wherein a ratio of the D-lactate produced as a product or byproduct of a metabolic process of live Lactobacillus crispatus cells to added lactate is from about 2:5 to about 2:1.

10. The postbiotic composition of claim 9, wherein the added lactate is selected from the group consisting of lactate salts, lactic acid, alkyl lactates, and combinations thereof.

11. The postbiotic composition of claim 1, further comprising maltose.

12. A therapeutic formulation, comprising:
the postbiotic composition of claim 1; and
a pharmaceutically acceptable vehicle.

13. The therapeutic formulation of claim 12, configured for intravaginal or topical administration.

14. A method for treating, preventing, or reducing the likelihood of a disease, disorder, condition, or symptom in a human subject, comprising administering to the subject a therapeutically effective amount of the postbiotic composition of claim 1.

15. The method of claim 14, wherein the disease, disorder, condition, or symptom is selected from the group consisting of bacterial vaginosis, pelvic inflammatory disease, sexually transmitted infection, postpartum endometritis, postpartum sepsis, preterm birth, preterm premature rupture of membranes, miscarriage, chorioamnionitis, intra-amniotic infection, infertility, ineffectiveness of infertility treatment, cervical intraepithelial neoplasia, cervical cancer, another cancer of the female genitourinary system, and combinations thereof.

16. A method for manufacturing a postbiotic composition, comprising:
culturing Lactobacillus crispatus cells in a fermentation medium, whereby the Lactobacillus crispatus cells produce D-lactate as a product or byproduct of a metabolic process;
separating at least a portion of the Lactobacillus crispatus cells and at least a portion of the D-lactate from the fermentation medium;
inactivating the separated Lactobacillus crispatus cells; and
combining the inactivated Lactobacillus crispatus cells and the separated D-lactate to form the postbiotic composition.

17. The method of claim 16, wherein the inactivating step comprises at least one of heat inactivation, ultraviolet inactivation, chemical treatment, gamma irradiation, sonication, and tabletization.

18. The method of claim 16, wherein, in the combining step, the inactivated Lactobacillus crispatus cells and the separated D-lactate are further combined with an added source of lactate.

19. The method of claim 18, wherein an amount of lactate provided by the added source of lactate is about 50% to about 250% of the amount of the separated D-lactate.

20. The method of claim 18, wherein the added source of lactate is selected from the group consisting of lactate salts, lactic acid, alkyl lactates, and combinations thereof.
